(19) United States
(12) Patent Application Publication
(10) Pub. No.: US 2013/0273168 A1
(43) Pub. Date: Oct. 17, 2013

(54) SOLID DOSAGE FORM COMPRISING PROTON PUMP INHIBITOR AND SUSPENSION MADE THEREOF

(71) Applicant: AstraZeneca AB, Sodertalje (SE)

(72) Inventors: Eva PERSSON, Lomma (SE); Eva TROFAST, Lund (SE)

(21) Appl. No.: 13/910,780

(22) Filed: Jun. 5, 2013

Related U.S. Application Data
(63) Continuation of application No. 11/312,869, filed on Dec. 19, 2005, now abandoned.
(60) Provisional application No. 60/638,435, filed on Dec. 22, 2004.

(51) Int. Cl. A61K 9/16; A61K 9/00; A61K 31/4439

(52) U.S. Cl. CPC A61K 9/1652; A61K 31/4439; A61K 9/1623; A61K 9/0053; A61K 9/1682
USPC 424/490; 514/338; 514/777

(57) ABSTRACT

A solid, rapidly gelling oral pharmaceutical dosage form, as well as an aqueous formulation prepared thereof, comprising a) an acid sensitive proton pump inhibitor as active ingredient distributed in a multitude of enteric coated pellets, and b) a suspension modifying granulate. Furthermore, the invention relates to an improved process for the manufacture and the use of such formulation in medical treatment, including prevention of gastrointestinal disorders in humans.

This application claims the benefit of U.S. Provisional Application No. 60/638,435, filed Dec. 22, 2004.

### FIELD OF THE INVENTION

This invention relates to a solid rapidly gelling oral pharmaceutical dosage form, as well as an aqueous formulation prepared thereof, comprising (a) an acid sensitive proton pump inhibitor as active ingredient distributed in a multitude of enteric coated pellets and (b) a to suspension modifying granulate. Furthermore, the invention relates to an improved process for the manufacture and the use of such formulation in medical treatment, including prevention of gastrointestinal disorders in humans.

### BACKGROUND OF THE INVENTION

Proton pump inhibitor (in the following also designated as “PPI”) compounds useful as H⁺K⁺-ATPase inhibitors include compounds having the generic names of omeprazole, lansoprazole, pantoprazole, rabeprazole, tenatoprazole and esomeprazole.

These active substances are useful for inhibiting gastric acid secretion in mammals and man. In a more general sense, they may be used for prevention and treatment of gastric acid related diseases in mammals and man, including e.g. reflux esophagitis, gastritis, duodenitis, gastric ulcer and duodenal ulcer. Furthermore, they may be used for treatment of other gastrointestinal disorders where gastric acid inhibitory effect is desirable, e.g. in patients on NSAID therapy, in patients with Non Ulcer Dyspepsia, in patients with symptomatic gastro-esophageal reflux disease, and in patients with gastrinomas. They may also be used in patients in intensive care situations, in patients with acute upper gastrointestinal bleeding, pre- and postoperatively to prevent acid aspiration of gastric acid, and to prevent and treat stress ulceration. Further, they may be useful for prevention and treatment of irritable bowel syndrome (IBS), inflammatory bowel disease (IBD), ulcerative colitis, Crohn's disease, asthma, laryngitis, Barret's syndrome, sleep apnea, sleep disturbance, and psoriasis, as well as being useful for prevention and treatment of Helicobacter infections, and diseases related to any of the above-mentioned conditions.

These active compounds are, however, susceptible to degradation/transformation in acidic and neutral media. The degradation is catalyzed by acidic compounds and is stabilized in mixtures with alkaline compounds. The stability of the active substances is also affected by moisture, heat, organic solvent content, and to some degree by light.

Oral dosage forms remain a significant problem for many patients, as many are unable or unwilling to swallow a solid dosage form. This problem occurs primarily in children and the elderly. It affects patient compliance, and is therefore a problem in therapy.

The need for an oral administration form, which avoids the swallowing difficulties associated with traditional tablets, has been recognized since many years. Syrups; elixirs, microcapsules containing slurries, and other novel tablet or capsule dosage forms have been developed. Among alternative forms for oral administration of pharmacologically active substances is the use of a solution or a suspension of the active ingredient in an aqueous medium.

Ready to consume suspensions (or solutions) have drawbacks associated with larger storage volumes and often limited shelf-life or the need for refrigerator storage. A particular problem that sometimes arises with aqueous suspensions is that some solid particles have a strong tendency of sinking to the bottom of the vessel used for administration. This may cause a part of the dose to be retained in the vessel and not the entire dose entering the oral administration route. Another problem that is sometimes experienced, is when using a suspension of particles in a liquid medium for administration through a nasogastric tube, the particles may tend to aggregate or agglomerate, thereby making it impossible for them to pass through the tube. Still another problem is when the liquid medium has a too high viscosity/viscoelasticity, which makes it impossible to administer the liquid medium through a nasogastric tube at a practical pressure.

It is a strong desire, particularly when administering acid-labile compounds such as proton pump inhibitors, for instance omeprazole, esomeprazole, pantoprazole and lansoprazole, to get an easily and quickly prepared, easily swallowable homogeneous suspension comprising the proton pump inhibitor in a form protecting it from contact with acidic environments, (e.g. the acidic gastric fluids). In addition, it is desirable that the suspension have viscoelastic properties and a viscosity suitable for allowing it to be administered via a gastric tube or to be swallowed. Furthermore, a liquid suspension formulation requires a certain viscosity to be stable over time.

With a drug preparation comprising water-insoluble components and which is to be stored as a dry powder mixture, and which is intended to be given as an ex-tempore prepared is homogeneous liquid suspension, other challenges/problems arise.

For some prior art compositions, there is a problem with viscosity in that a maximum viscosity level is obtained only after long hold times, i.e. the viscosity is not constant over the short time frames from the time when the suspension is made until the time the suspension is administered to the patient. There may also be problems with batch-to-batch variation regarding the amount of time required to obtain a stable maximum viscosity level in the suspension prepared from a dry powder mixture.

Intolerance to lactose-containing foods is a common problem. Thus, medicaments containing lactose may pose a problem for such people.

There are proposals in the art regarding compositions comprising a proton pump inhibitor, and there are other proposals relating to methods for quickly dispersing and/or dissolving formulations.

U.S. Pat. No. 5,731,002 describes a stable, oral pharmaceutical composition comprising a proton pump inhibitor in a paste-like gel designed for the treatment of gastric acid related diseases in animals.

U.S. Pat. No. 5,840,737 discloses a method for treating gastric acid disorders with compositions comprising omeprazole or lansoprazole together with bicarbonates.

Problems associated with administering bicarbonates such as sodium- or potassium bicarbonate to patients such as humans include belching that may result when the carbonate is neutralized in the stomach. Patients with gastroesophageal reflux may exacerbate or worsen their disease as the belching can cause upward movement of stomach acid (Brunton, Agents for the control of gastric acidity and treatment of peptic ulcers. In: Goodman, A. G. et al. The pharmacologic basis of therapeutics, p. 907, New York, 1990). Moreover, there is a possibility that intake of sodium bicarbonate may cause metabolic alkalosis.

There are further published patent applications in the same patent family, such as US 2002/0045646 A1, which discloses a solid non-enteric coated dosage form compositions comprising a proton pump inhibitor and a buffer. Other applications in the family, such as US2003/118669, US2003/144306, US2003/191159, US2003/215527, US2004/048896 and US2004/171646, disclose, for instance, liquid oral pharmaceutical compositions comprising a proton pump inhibitor and a buffering agent and a method of increasing absorption of the proton pump inhibitor.

US 2004/0005362 A1 (Taneja) and US 2004/0082618 A1 (Taneja) describe a pharmaceutical formulation comprising an acid labile drug coated with an enteric coating and a liquid vehicle of pH less than 6.0. Other published applications from the same inventor describe, for instance, a liquid vehicle of a viscosity sufficient to suspend microgranules comprising a PPI (US 2004/0081700 A1) or (US 2004/0006109 A1 and US 2004/0081671 A1) wherein the pH of the liquid vehicle is greater than 6.5.

WO 2004/004690 A1 (Taneja) discloses a liquid dosage form having enteric coated microgranules comprising an acid-labile drug and a liquid suspension having a pH less than 6.0 and a viscosity sufficient to suspend the microgranules. Carbonates or bicarbonates may be used in the dosage forms.

US 2004/0022854 A1 discloses an oral administration form for acid-labile active compounds wherein the auxiliaries are not suitable for formation of enteric layers (enteric coating). Prepared active compound units can be formulated into sachets, e.g. together with lactose, or formulated together with carbonate containing excipients to provide an effervescent composition.

EP 1,232,746 describes a readily suspendible dry powder mixture composition comprising a gellant or thickener. The thickener comprises at least one xanthan gum having a specific particle size distribution, a filler, a wetting agent or surfactant, and a pharmacologically active substance.

U.S. Pat. No. 4,886,669 describes a water-dispersible tablet comprising a pharmaceutically active agent, at least one disintegrant, and a swellable material. It is stated that the tablet disintegrates rapidly in water forming a homogeneous suspension of high viscosity that can easily be swallowed.

U.S. Pat. No. 5,008,117 relates to a method for preparing a quickly dispersing and dissolving formulation of thickening or suspending agents and other excipients, in which drug microcapsules are readily dispersible. Proton pump inhibitors are not mentioned.

EP 0491910 B1 discloses a solid pharmaceutical composition for addition to water to produce a suspension of a drug. The composition comprises a thickening or suspending agent, an acid, and a carbonate or bicarbonate.

U.S. Pat. No. 6,261,602 describes a granular composition useful as a pharmaceutical carrier which can be used for the preparation of pharmaceutical compositions that are capable of rapid suspension in water or aqueous media. The composition may be prepared by a process which comprises subjecting a mixture of a thickening agent and a disintegrating agent to wet granulation with an aqueous medium as wetting agent, or to dry granulation to make a granular product.

### BRIEF DESCRIPTION OF THE INVENTION

The present invention avoids the above discussed disadvantages with prior art compositions and presents a solution to the previous mentioned problems. It further provides a mean for making a drug vehicle which is suitable for administration via a gastric tube due to good viscosity and viscoelasticity properties of the obtained vehicle (suspension). The vehicle is robust enough to provide approximately the same viscosity even if the amount of water used varies within 50% to 150% of the prescribed amount.

The present invention relates to a solid rapidly gelling oral pharmaceutical dosage form comprising (a) an acid sensitive proton pump inhibitor compound as active ingredient distributed in a multitude of enteric coated pellets and (b) a suspension modifying granulate.

Furthermore, it has now been surprisingly found that a special composed granulate can advantageously be mixed with a multitude of enteric coated pellets comprising a proton pump inhibitor. The granulate, when suspended in water, quickly and reproducibly will create an aqueous vehicle having a desired pH, a desirable stable viscosity level and a satisfactory viscoelasticity. This granulate is in the following also referred to as a “suspension modifying granulate”. Furthermore, this granulate should be free from bicarbonate and carbonate salts. According to one embodiment of the invention, it is possible to make this granulate free from lactose, and thereby tolerable for people having an intolerance to lactose.

The dosage forms of the invention render the quick formation of a viscous stable suspension possible. Prior to administration, the solid dry suspension modifying granulate and the enteric coated pellet are dissolved/suspended in an aqueous liquid, such as tap water, thereby providing a viscous liquid formulation for oral administration. When a dosage form of the invention is to be administered to the patient, it is important that the preparation be dissolved/suspended as quickly as possible, and at the same time provide a homogeneous suspension having a uniform distribution of the solid particles containing the pharmacologically active ingredient. Therefore, the final liquid formulation should ensure that practically all of the dose, even if the dose is comprised in a suspended, particulate form, is delivered to the oral cavity of a patient in a safe, reliable, and reproducible way.

When the active ingredient is comprised in enteric coated pellets, it is necessary that the suspension medium has a pH that does not cause premature dissolution of the enteric coating layer of the pellets comprising the active ingredient. In addition, the administration through naso-gastric tubes puts demands on the final liquid formulation regarding features such as suitable and stable viscosity, viscoelastic properties, and absence of agglomeration tendencies of the suspended particles.

A further feature is that the suspension is suitable for administration through thin tubes intended for pediatric use. The expression “gastric tube” includes naso-gastric tubes as well as any other tubes or syringes intended for feeding a suspension or dispersion into the stomach of a patient.

The viscoelastic and viscosity properties of the suspension become especially important as tubes used in pediatric treatment may have a narrow inner diameter and thereby being unsuitable for liquids which produce high back-pressures upon administration. One such example of a tube with narrow inner diameter is “Infant feeding tube, FT 1606/105 (CH/FG 6—2.0 mm outer diameter, 1.4 mm inner diameter), Pennine Healthcare.”

The dosage forms of the invention gel more rapidly in water at room temperature than prior art formulations to yield a homogeneous stable dispersion. Thus, they give a stable viscosity in less time than the prior art and, furthermore, they are robust with respect to the obtained viscosity properties.

In brief, the dosage forms of the invention comprise two principal components: a) a suspension modifying granulate and b) a multitude of enteric coated pellets comprising the active ingredient.

The suspension modifying granulate comprises:
a rapidly dissolving diluent;
a gelling agent;
an acidic pH-regulating agent;
a binder; and an optional disintegrant, and
   furthermore, the granulate is free from bicarbonate salts and/or carbonate salts.

According to one embodiment, the above described suspension modifying granulate is free from lactose. This further advantage makes it suitable for people suffering from lactose-intolerance who can therefore be treated with such embodiments of the invention.

One of the features of the invention is that the rapidly dissolving diluent is brought into close/intimate contact with the gelling agent. Not only does this give a very rapid gelling time compared to the gelling agent per se, it also very quickly yields a stable gel. According to another embodiment of the invention, the diluent may also function as a sweetener.

According to one feature of the invention, the rapid disintegration and quick gelling to obtain a stable and reproducible viscosity level when the suspension modifying granulate is suspended in water, is achieved by a special manufacturing process. According to this feature, the process comprises mixing together and granulating the gelling agent and diluent/sweetener together, and subsequently drying the obtained suspension modifying granulate to obtain a low moisture and/or solvent content.

Manufacture of the enteric coated pellets is described in the section “Detailed description of the invention”, but they can in general be manufactured according to directives in WO 96/01624 A1, taking into consideration any special requirements regarding the size of the pellets. Furthermore, there is no need for any “overcoat” on the enteric coated pellets.

The present invention provide safe and reliable dosage forms for oral or gastric-tube administration of enteric coated pellets comprising acid-labile proton pump inhibitors such as omeprazole, esomeprazole, pantoprazole, and lansoprazole dispersed in an aqueous liquid medium. Such administration is especially suitable and advantageous in the treatment of children or elderly patients.

The compositions of the present invention also allow the incorporation of a wide range of dosage levels and additional agents such as taste masking/improving agents and tonicity agents.

### BRIEF DESCRIPTION OF THE FIGURES

FIG. 1 shows viscosity versus time for an embodiment of the invention. (5 samples)

FIG. 2 shows viscosity versus time for a prior art embodiment (Lanzo™, 4 samples).

### DETAILED DESCRIPTION OF THE INVENTION

One aspect of the present invention is a dosage form comprising a mixture of a first component (I) which is a multitude of enteric coated pellets, and a second component (II) component which is a suspension modifying granulate, the mixture being dispensed in a container such as a sachet. The mixture rapidly disintegrates and gels when suspended in an aqueous medium, such as tap water, thus forming a homogeneous stable and robust suspension having a reproducible and stable viscosity. The suspension can easily be swallowed by the patient or administered nasally through e.g. a naso-gastric tube. The ready-to-use liquid suspension formulation provides a further aspect of the present invention and comprises three components, the two components (I) and (II) mentioned above, and in addition the liquid medium (III).

The rapid gelling, i.e. the short gelling time, of the present invention can be understood as the time required before substantially all of the enteric coated pellets in the prepared suspension remain suspended in the liquid medium and not sink to the bottom of the vessel (such as a glass or beaker) used for its preparation. The gelling time required for embodiments of the invention is in general shorter than 3 minutes, and preferably less than 2 minutes, when tested as described in Example 5.

The dosage form is free from bicarbonate salts and/or carbonate salts. One embodiment of the invention is furthermore free from lactose. “Free from” means that no such compound is added in the formulation. Trace amounts present in and accompanying other raw materials used in the composition are not taken into account by this expression.

### Enteric Coated Pellets

The enteric coated pellets comprising the active ingredient are manufactured with the outermost layer being the enteric coating layer. Such pellets can be manufactured according to methods known in the art, e.g. as described in WO 96/01624 A1, taking into consideration any special requirements regarding the size of the pellets. Furthermore, there is no need for any “overcoat” on the prepared enteric coated pellets.

According to one aspect of the invention, the average diameter of the enteric coated pellets is in the range of 0.2-1.8 mm, preferably 0.4-1.0 mm, and more preferably 0.5-0.8 mm.

In another aspect of the invention, the enteric coated pellets have a diameter which is in the range of 1.0-1.4 mm.

The enteric coated pellets are consisting of the following structural components;
a core material comprising the active ingredient,
an optional separating or subcoating layer, and
an enteric coating layer,
   but no additional coating layer.

### Core Material.

The core material is manufactured by processes known in the art, such as extrusion-spheronization, layering techniques such as powder- or solution/suspension layering, spray drying, balling, congealing techniques, or spray congealing techniques.

The core material comprises the active ingredient and may also comprise seeds, binders, surfactants, fillers, disintegrating agents, alkaline additives, or other pharmaceutically acceptable ingredients, alone or in mixtures.

### Active Ingredient

The pharmaceutical formulations of the invention comprise an acid sensitive proton pump inhibitor or an alkaline salt thereof or a single enantiomer or an alkaline salt of its enantiomer as active ingredient. The single enantiomers, racemic mixtures (50% of each enantiomer), and unequal mixtures of the two enantiomers are suitable for the pharmaceutical formulation according to the present invention.

The active ingredient, optionally together with excipients, is provided in small enteric coated pellets/beads.

Compounds/active ingredients of interest for the novel pharmaceutical compositions according to the present invention are compounds of the general formula I, an alkaline salt thereof, one of the single enantiomers thereof, or an alkaline salt of one of the enantiomers

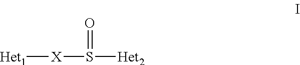

wherein

### Het₁ is

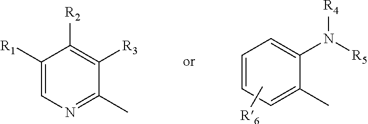

### Het₂ is

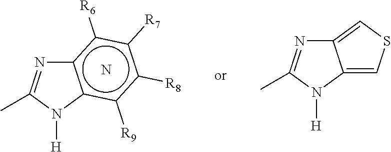

### X=

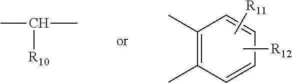

wherein
N in the benzimidazole moiety means that one of the ring carbon atoms substituted by R₆-R₉ optionally may be exchanged for a nitrogen atom without any substituents;
R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;
R₄ and R₅ are the same or different and selected from hydrogen, alkyl and arylalkyl;
R₆′ is hydrogen, halogen, trifluoromethyl, alkyl or alkoxy;
R₆-R₉ are the same or different and selected from hydrogen, alkyl, alkoxy, halogen, halo-alkoxy, alkylcarbonyl, alkoxycarbonyl, oxazolinyl, pyrrolyl and trifluoroalkyl, or adjacent groups R₆-R₉ form ring structures which may be further substituted;
R₁₀ is hydrogen or forms an alkylene chain together with R₃ and
R₁₁ and R₁₂ are the same or different and selected from hydrogen, halogen and alkyl.

In the above definitions alkyl groups, alkoxy groups and moities thereof may be branched or straight C₁-C₉-chains or comprise cyclic alkyl groups, for example cycloalkylalkyl.

Examples of specifically interesting compounds according to formula I are

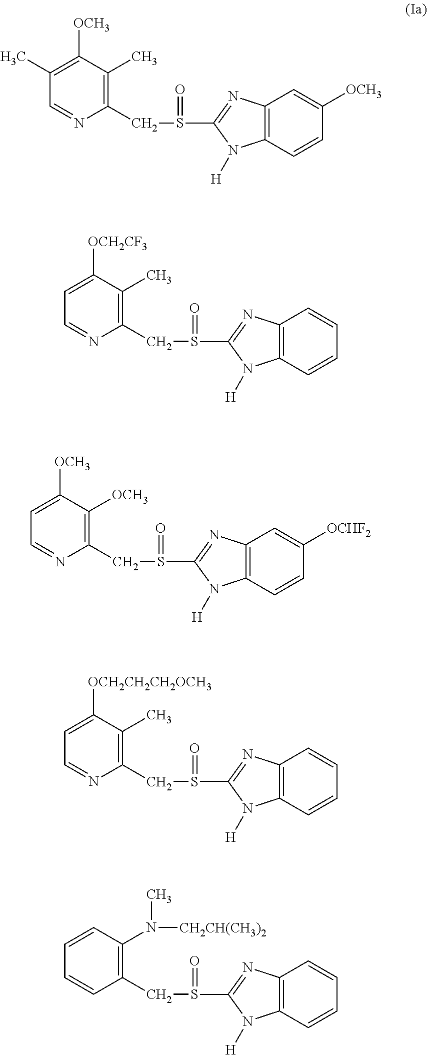

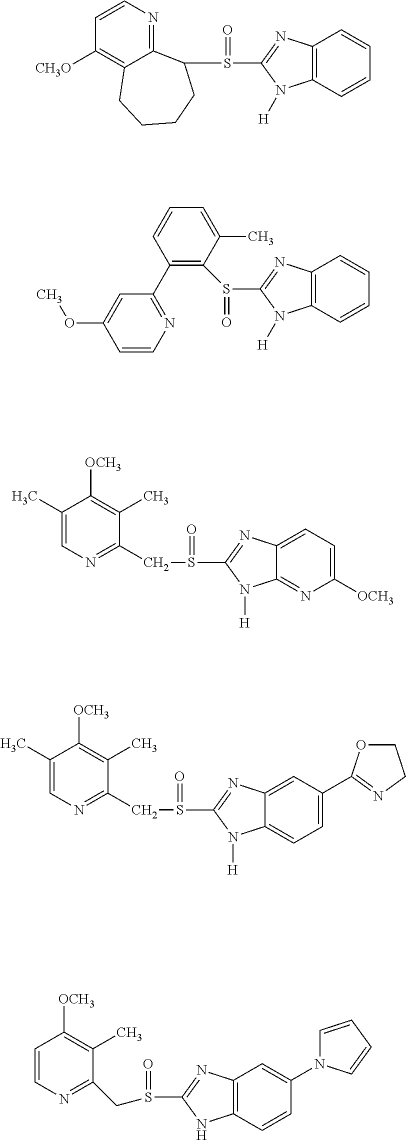

including tautomeric forms thereof.

Preferred compounds for the oral pharmaceutical preparation according to the present invention are omeprazole, a magnesium salt of omeprazole, or a magnesium salt of the (−)-enantiomer of omeprazole. The last-mentioned compound has the generic name of esomeprazole.

According to one embodiment, the active ingredient is esomeprazole magnesium trihydrate.

In another embodiment of the invention, tenatoprazole, a pharmaceutically acceptable salt of tenatoprazole, a single enantiomer of tenatoprazole, or a pharmaceutically acceptable salt of the single enantiomer, is the active drug.

According to another aspect of the invention, the active ingredient is a hydrated form of any one of the aforementioned compounds.

In one aspect of the invention, the amount of active ingredient in the preparation is in the range of 1 mg-100 mg, 2 mg-80 mg, or 5 mg-50 mg.

### Seeds

The seeds which are to be layered with the active substance can be water-insoluble seeds comprising different oxides, celluloses, organic polymers, and other materials, alone or in mixtures; or water soluble seeds comprising different inorganic salts, sugars (excluding lactose), non-pareils and other materials, alone or in mixtures. Further, the seeds may comprise the active substance in the form of agglomerates, compacts, etc.

### Binders

Examples of binders which can be used in the present invention include celluloses such as hydroxypropyl methylcellulose, hydroxypropyl cellulose and carboxymethyl-cellulose sodium, polyvinyl pyrrolidone, polyethylene glycols, polyvinyl alcohols, sugars (excluding lactose), starches, and other pharmaceutically acceptable substances with cohesive properties.

### Surfactants

Surfactants may be used in the dosage form. Suitable surfactants include pharmaceutically acceptable non-ionic surfactants, such as Polysorbate 80, and ionic surfactants such as sodium lauryl sulfate.

### Fillers

Fillers may be used in the dosage form. Examples of fillers include mannitol and dicalcium phosphate.

### Disintegrating Agents

A disintegrating agent may be used in the dosage form. Examples of disintegrating agents is that can be used are cross-linked polyvinyl pyrrolidone, pregelatinized starch, microcrystalline cellulose, and cross-linked sodium carboxymethyl cellulose.

### Alkaline Additives

According to one embodiment of the invention, the active substance may also be mixed with an alkaline pharmaceutically acceptable substance (or substances). Such substances can, after excluding bicarbonate salts or carbonate salts, be chosen among, but are not restricted to, substances such as the sodium, potassium, calcium, magnesium, and aluminium salts of phosphoric acid, citric acid, or other suitable weak inorganic or organic acids; substances normally used in antacid preparations such as aluminium, calcium and magnesium hydroxides; magnesium oxide; organic pH-buffering substances such as trihydroxymethylaminomethane, basic amines, or amino acids and their salts, and other similar, pharmaceutically acceptable pH-buffering substances.

### Separating or Subcoating Layer

The separating or subcoating layer(s) can be applied to the core material by coating or layering procedures using suitable equipment, such as coating pans, coating granulators, or in a fluidized bed apparatus using water and/or organic solvents for the coating process. As an alternative, the separating layer(s) can be applied to the core material by using a powder coating technique. The materials for the separating layer(s) are pharmaceutically acceptable compounds, such as sugar, polyethylene glycol, polyvinylpyrrolidone, polyvinyl alcohol, polyvinyl acetate, hydroxypropyl cellulose, methyl-cellulose, ethylcellulose, hydroxypropyl methyl cellulose, carboxymethylcellulose sodium, and others, used alone or in mixtures. Additives such as plasticizers, colorants, pigments, fillers, anti-tacking and anti-static agents, such as magnesium stearate, titanium dioxide, fumed silica, talc, and other additives may also be included into the separating layer(s).

The separating layer(s) may serve as a diffusion barrier and may act as a pH-buffering zone. The pH-buffering properties of the separating layer(s) can be further strengthened by introducing into the layer(s) substances, after excluding bicarbonate salts or carbonate salts, chosen from a group of compounds usually used in antacid formulations, such as magnesium oxide, magnesium hydroxide, and aluminium or calcium hydroxide or silicate; composite aluminium/magnesium compounds, such as MgO.Al₂O₃.2SiO₂.nH₂O; and other pharmaceutically acceptable pH-buffering compounds, such as the sodium, potassium, calcium, magnesium and aluminium salts of phosphoric, citric or other suitable, weak, inorganic or organic acids; or suitable organic bases, including basic amino acids or amines and salts thereof. Talc or other compounds may be added to increase the thickness of the layer(s) and thereby strengthen the diffusion barrier.

### Enteric Coating Layer

One or more enteric coating layers are applied onto the core material, or onto the core material covered with separating layer(s), by using a suitable coating technique. The enteric coating layer material may be dispersed or dissolved in either water or in suitable organic solvents. As enteric coating layer polymers, one or more, separately or in combination, of the following substances can be used: solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, and other suitable enteric coating layer polymer(s).

The enteric coating layers can contain pharmaceutically acceptable plasticizers to obtain the desired mechanical properties, such as flexibility and hardness of the enteric coating layers. Such plasticizers include, but not restricted to, triacetin, citric acid esters, phthalic acid esters, dibutyl sebacate, cetyl alcohol, polyethylene glycols, polysorbates, and other plasticizers.

### Suspension Modifying Granulate.

The suspension modifying granulate comprises:
a rapidly dissolving diluent;
a gelling agent;
an acidic pH-regulating agent;
a binder; and
optionally a disintegrant,
   and in addition, the suspension modifying granulate is free from bicarbonate salts or carbonate salts, and especially from components that can result in effervescence.

According to one embodiment, the suspension modifying granulate is manufactured by a process in which the rapidly dissolving diluent and the gelling agent are mixed and granulated together, and thereafter dried.

The final moisture content in the suspension modifying granulate measured as loss on drying is less than 3% (w/w), and preferably less than 1% (w/w). The final content of ethanol is less than 0.2% (w/w), and preferably less than 0.12% (w/w).

When the suspension modifying granulate is suspended in tap water, a stable and close-to-maximum viscosity is obtained in a short time. Further, the suspension obtained is free from lumps and is robust, in the sense that its viscosity properties are approximately the same even if a patient adds too little or too much water when preparing the suspension from the granulate. Thus, it is possible to add one dose of the active ingredient and the suspension modifying granulate to 50%-150% of the prescribed amount of water and still obtain the desired properties of the formulation.

The gel formed when adding the suspension modifying granulate to an aqueous medium, such as water, has a viscosity of 3.0 to 6.0 log (mPas)=10³ to 10⁶ mPas, and preferably 3.6 to 4.7 log (mPas)=10^{3.6} to 10^{4.7} mPas.

This viscosity is determined at 20° C. from the intercept at the viscosity axis of the line when plotting log(viscosity) against log(rotational speed (rpm)). The line is made by a linear fit using least square linear regression, and the intercept of the fitted line is determined. Suitable equipment for determination of viscosity is used, such as a Physica DV-1 P viscometer, using a No. 2 spindle, 18.7 mm in diameter, length 6.9 mm, which is operated at rotational speed 3.0, 6.0, 30, and 100 rpm. Measurements are made until a stable value is obtained (about 1 minute).

The rapid disintegration and quick gelling to yield a stable and reproducible viscosity level is achieved, according to one aspect of the invention, when a special manufacturing process is used for preparing the suspension modifying granulate.

This manufacturing process includes the following steps:
I) mixing the gelling agent with the pH-regulating agent, the rapidly dissolving diluent, and the optional disintegrant;
II) dissolving the binder in ethanol;
III) wetting the mixture obtained in step I with the solution obtained in step II;
IV) agitating the wet mixture obtained in step III such that substantially each particle of the gelling agent is in contact with the rapidly dissolving diluent;
V) drying the agitated wet mixture from step IV until the final moisture content in the granulate measured as loss on drying is less than 3% (w/w); and
VI) grinding or milling the dry granules obtained in step V until more than 95% (w/w) of the granules pass through a sieve having 1.0 mm openings.

Alternatively, step II in the above process can be performed before step I.

One feature of the invention is to bring the rapidly dissolving diluent into close/intimate contact with the gelling agent, thereby not only giving a very rapid gelling time compared to the gelling agent by itself, but also very quickly providing a stable gel. One embodiment of the invention provides for the selection of a suitable rapidly disintegrating diluent, which also may function as a sweetener.

In general, the gelling of the dry suspension modifying granulate when added to water, such as tap water, is rapid, for example, reaching 75% of the maximum obtainable level already within approx. 10 minutes. 90% or more of the maximum viscosity is generally reached within 15 minutes. Comparative data is provided in the Table in Example 2.

In one embodiment of the invention, when a suspension modifying granulate according to the invention is suspended in water and mildly agitated, a suspension is obtained having, within 13 minutes, a viscosity which is at least 75% of the maximum obtainable viscosity, and preferably more than 75% of the maximum obtainable viscosity within 10 minutes, as tested with 1 g of suspension modifying granulate added to 5 ml of water. In another embodiment of the invention, more than 90% of the maximum obtainable viscosity is reached within 30 minutes, and preferably more than 90% of the maximum obtainable viscosity is obtained within 25 minutes, as tested with 1 g of suspension modifying granulate added to 5 ml of water.

According to one embodiment of the invention, the suspension modifying granulate (and the enteric coated PPI comprising pellets) does not contain lactose.

### Gelling Agent

The gelling agent provides for forming a gel suitable for administration through a gastric sond/naso-gastric tube, and is chosen to have the proper viscoelasticity as well as the proper viscosity of the gel formed when dispersed in an aqueous medium, such as water. This is a desired administration route in pediatric or geriatric therapy.

The dissolution time will also influence the selection of gelling agents.

Suitable gelling agents of the invention are different qualities of xanthan gums.

Other gelling agent can be considered, but in the case of certain gelling agents, such as starch products, for example, Thick-It™ regular, containing modified corn starch and maltodextrin, the suitable range of concentrations is very limited. This product can generally be used only in the narrow range of about 6 to 8% of the final suspension, to corresponding to a content of gelling agent in the suspension modifying granulate of 34 to 48%, which is an unsuitably high proportion of the composition.

Another example is corn starch, that many times will give rapid swelling, but has undesired viscoelastic properties.

Gelling agents like Na-carboxymethylcellulose (CMC) and carrageenan could not be used is in the present invention due to lack of suitable viscoelastic properties or due to unsuitable properties for administering the obtained suspension through a gastric sond.

Thus, gelling agents in the invention are chosen among xanthan gums.

The concentration of the gelling agent is 0.6 to 12% (w/w) of the suspension modifying granulate. In a preferred embodiment, the concentration of the gelling agent is between 1.8 to 4.8% (w/w) of the suspension modifying granulate. A concentration of the gelling agent in this range has practical utility for the patient and provides suitable properties of the viscoelestic gel.

In one embodiment of the invention, the gelling agent has an average particle size larger than 150 microns.

### Rapidly Dissolving Diluent

The diluent has a diluting function but it may also function as a sweetener.

The diluent is selected from the group consisting of monosaccharides, hydrates of monosaccharides, disaccharides, and hydrates of disaccharides. According to one aspect of the invention preferred diluents are glucose, hydrates of glucose, sucrose, and hydrates of sucrose. According to the present invention, rapid dissolution signifies that the dissolution time of the diluent is below 2 minutes when 2 g of the substance is dissolved in 10 ml of water during slow continuos stirring at 14° C. One diluent specifically not fulfilling this requirement is mannitol.

As a consequence of the manufacturing method, the suspension modifying granulate according to the invention has the rapidly dissolving diluent randomly distributed throughout the obtained individual granulate particles.

Acidic pH-Regulating Agent

The pH of the suspension modifying granulate when suspended in water should be in the range of between 3.0 and 6.0, preferably in the range of between 3.0 and 5.0, and more preferably in the range of between 3.5 and 4.5.

This pH may be achieved by adding a suitable acidic pH-regulating agent. This agent may consist of a single acidic chemical compound, or a mixture of compounds chosen among acidic and alkaline compounds, with the exception of any carbonate salts. Any mixture of such pH influencing compounds is chosen in such a way that when the mixture is dissolved/suspended in water, it will give a pH within the desired (acidic) range as discussed above.

Non-limiting examples of suitable acidic compounds are citric acid, tartaric acid, and malic acid. A non-limiting example of a mixture of compounds chosen among acidic and alkaline compounds is monosodium phosphate and disodium phosphate (in an appropriate ratio to achieve a pH within the desired range).

### Disintegrant

The optional disintegrant used in the dry suspension modifying granulate may be a single disintegrant or a mixture of disintegrants.

Non-limiting examples of suitable disintegrants include cross-linked polyvinyl pyrrolidone, crosslinked sodium carboxymethyl cellulose (Ac-Di-Sol®), and pregelatinized starch (Sta-Rx® 1500).

### Binder

An example of a suitable binder used according to the present invention is a polymer that is soluble in water and in ethanol. Suitable binders include selected qualities of hydroxypropylcellulose.

When the binder is a hydroxypropyl cellulose (in the following also referred to as HPC), it typically has a hydroxypropyl content in the range of 50-90%, or more preferably in the range of 60-81%, and a viscosity below 450 mPas (cps) tested at 5% concentration. Such is a polymers are, for example, Klucel® JF and Klucel® LF from Aqualon.

The hydroxypropyl celluloses contemplated for use in this aspect of the invention, as a binder, do not include low-substituted hydroxypropyl cellulose, also referred to as L-HPC.

The ratio between the binder and the gelling agent in the suspension modifying granulate of the invention is preferably in the range of from 1:2 to 1:3 (w/w).

### Dosage Form Strengths

Different product dosage form strengths are obtained by filling specific amounts of enteric coated proton pump inhibitor pellets and the suspension modifying granulate of the invention into unit size sachets. According to one embodiment of the invention, the enteric coated pellets comprise esomeprazole magnesium trihydrate and are combined with the suspension modifying granulate into unit size sachets.

The ratio (w/w) between the two components of the mixture—i.e. between the enteric coated pellets comprising the proton pump inhibitor on the one hand, and the (dry) suspension modifying granulate on the other hand—may vary between 1:1000 to 100:1000, preferably between 4:1000 to 80:1000, and most preferably between 8:1000 to 60:1000.

### Amount of Enteric Coated Pellets in One Sachet.

The enteric coated pellets comprising the proton pump inhibitor have a drug content from 5% (w/w) of the enteric coated pellets to 40% (w/w) of the enteric coated pellets. This means that the highest theoretical amount of pellets for one dose, calculated for the lowest concentration of drug for the highest dose of drug (100 mg acc. to invention), gives a total of (100/0.05=) 2000 mg pellets.

A similar calculation of the lowest possible amount of pellets for one dose, using the highest concentration and the lowest dose (1 mg acc. to the invention), gives the minimum amount of pellets as (1/0.4=) 2.5 mg pellets.

In a preferred embodiment of the invention, the drug content of the enteric coated pellets is 8-30% (w/w).

The amount of enteric coated pellets in one sachet according to the invention is in the range of 2.5-2000 mg, and in the preferred embodiment of the invention, the amount of enteric coated pellets in one sachet is in the range of 3-1250 mg.

In an alternative embodiment of the invention, the drug content of the enteric coated pellets in one sachet is determined according to the following table:

TABLE 1

| | Adapted drug | Amount of |
|---|---|---|
| Intended dose | content in the | pellets in one |
| in one sachet | enteric coated pellets | sachet |
| 1 mg-40 mg | 8-12% w/w | 8-500 mg |
| >40 mg-70 mg | 15-25% w/w | 160-467 mg |
| >70 mg-100 mg | 25-40% w/w | 280-400 mg |

Thus, in one embodiment of the invention, the dose in one sachet is 1-40 mg and the drug content in the enteric coated pellets is 8-12% (w/w).

In another embodiment of the invention, the dose in one sachet is greater than 40 mg-70 mg and the drug content in the enteric coated pellets is 15-25% (w/w).

In a further embodiment of the invention, the dose in one sachet is greater than 70 mg-100 mg and the drug content in the enteric coated pellets is 25-40% (w/w).

### Ready-for-Use Liquid Formulation

Prior to use, the content of the sachet is emptied into a predefined volume of an aqueous liquid. After stirring, a viscous suspension is formed. This liquid formulation is another aspect of the invention, and consists of three main components: a) enteric coated pellets comprising the proton pump inhibitor, b) (dry) suspension modifying granulate, and c) an aqueous liquid.

The amount of the aqueous liquid is intended to be 5 times the amount of suspension modifying granulate, but the invention allows for the patient to vary this amount of aqueous liquid from 50% up to 150% of the prescribed amount. This means that the amount of the aqueous liquid in the ready-for-use liquid formulation is in the range of from 2.5 times to 7.5 times of the amount of the suspension modifying granulate.

In one embodiment of the invention, the aqueous liquid is water.

The concentration of the gelling agent should be 0.1 to 2% (w/w) (a twenty-fold range in concentration) of the suspension, preferably between 0.3 to 0.8% (w/w). Advantageously, this broad range in concentration of the gelling agent provides practical utility for the patient while still maintaining relevant properties of the viscoelastic gel.

### EXAMPLES

### Example 1a

### Preparation of the Suspension Modifying Granulate According to the Invention

| Excipient | Content (%) |
|---|---|
| Xanthan Gum 11K | 2.5 |
| Polyvinylpyrrolidone cross-linked | 2.5 |
| Glucose, water free | 93.8 |
| Hydroxypropyl cellulose JF | 1.0 |
| Citric acid anhydrous | 0.164 |
| Colour iron dioxide yellow | 0.06 |

The hydroxypropyl cellulose is dissolved in ethanol. The cellulose solution is added to a is dry mixture of the remaining excipients, giving a wet mass which is granulated during the addition of the solution. The wet mass is dried and ground (maximum 5% of the granules >1 mm diameter).

3 g of this suspension modifying granulate was dissolved in 15 ml water and the liquid formulation was stirred for 60 s. The pH was measured with a glass electrode using a calibrated pH meter and found to be 4.0.

### (Comparative) Example 1b

### Suspension Modifying Granulate According to Prior Art

Comparisons were made using the commercially product “Lanzo™ 30 mg, granulate” from Wyeth Lederle (batch 3ET032, expiry date July 2006; and batch 3ET010, expiry date March 2006).

According to the SWEDIS online medical database, the suspension granulate composition (excluding the enteric coated pellets) used in this product is as follows:

| Excipient | Content (%) |
|---|---|
| Mannitol | 45.8 |
| Sucrose | 45.8 |
| Xanthan gum | 3.5 |
| Polyvinylpyrrolidone, cross-linked | 3.5 |
| Dioctyl sulfosuccinate | 0.015 |
| Magnesium stearate | 0.5 |
| Silicon dioxide | 0.1 |
| Citric acid anhydrous | 0.4 |
| Color | 0.05 |
| Flavouring | 0.4 |

### Ex 2

### Viscosity Measurements

### Experimental Conditions

Embodiment according to the invention: 3 g of the suspension modifying granulate obtained according to Example 1a was dissolved in 15 ml water, and the liquid formulation was stirred for 60 s.

Prior art sample (Lanzo™ 30 mg, granulate): The lansoprazole-comprising pellets were removed from the total solids (5.7 g) of the product described in Example 1b, and to the remaining powder/granulate (5.4 g) was added 30 ml water, after which the liquid to formulation was stirred for 60 s.

For both samples, viscosity measurements started after another 1 min.

Instrument: Reologica Stresstech

Measuring principle: Oscillation with plate/plate P 30 2 mm slit

Measuring parameters: Frequency 0.1 Hz; stress 0.07146 Pa.

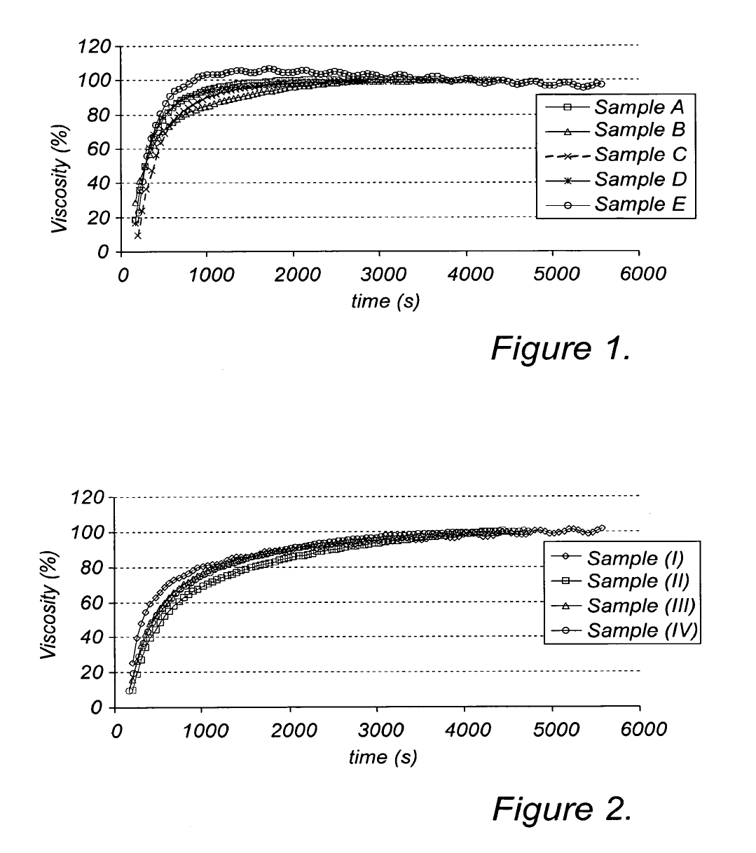

Time to arrive at Viscosity in % of Maximum Viscosity
(Evaluated from FIG. 1 and 2.)

| | Percentage of maximum Viscosity | |
|---|---|---|
| | >75% | >90% |
| Ex. 1a (invention) n = 5 | average = 9.7 min | average = 14.8 min |
| | min = 7.6 min | min = 9.5 min |
| | max = 12.6 min | max = 23.1 min |
| Ex. 1b (prior art) n = 4 | average = 16.8 min | average = 32.5 min |
| | min = 13.3 min | min = 29.0 min |
| | max = 21.2 min | max = 39.5 min |

### Discussion

In the case of lansoprazole (Ex. 1b), although the suspension granulation formulation has a fast dissolving diluent (sucrose) the formulation will not form a stable gel within the desired short time frame (see FIG. 2), as compared to the formulation which is obtained with the present invention (Ex. 1a) (see FIG. 1) as shown in the Table above.

The result of using a slowly dissolving diluent will be a composition with slower gelling times and a continously-increasing viscosity within a reasonable and adequate time period. Thus, the present invention has solved several problems in order to obtain a lactose-free and bicarbonate/carbonate-free composition having rapid gelling time with a viscosity/viscoelasticity suitable for swallowing or administration through a tube. The inventive composition has a constant viscosity over time, and no lumps are present in the final suspension to be administered.

### Example 3

### Manufacturing of Enteric Coated Pellets Comprising Esomeprazole-Mg-Trihydrate

| Core material | |
|---|---|
| Esomeprazole-Mg trihydrate | 445 g |
| Sugar sphere seeds | 300 g |
| Hydroxypropyl methylcellulose | 67 g |
| Polysorbate 80 | 9 g |
| Purified water | 2100 g |

| Subcoating layer | |
|---|---|
| Hydroxypropyl cellulose | 90 g |
| Talc | 340 g |
| Magnesium stearate | 22 g |
| Purified water | 3100 g |

| Enteric coating layer | |
|---|---|
| Methacrylic acid copolymer type C, | 1270 g |
| 30% dispersion | |
| Triethyl citrate | 38 g |
| Mono- and diglycerides | 19 g |
| Polysorbate 80 | 2 g |
| Purified water | 500 g |

Suspension layering was performed in a fluid bed apparatus using a bottom spray technique. Esomeprazole was sprayed onto the sugar sphere seeds from a water suspension containing the dissolved binder and surfactant. The size of the sugar spheres seeds were in the range of 0.25 to 0.35 mm.

The prepared core material was covered with a hydroxypropyl cellulose solution containing talc and magnesium stearate in a fluid bed apparatus to form the subcoating layer. The enteric coating layer was sprayed as a water dispersion onto the pellets covered with the separating layer in a fluid bed apparatus.

### Example 4

### Examples of Component Ratios for Preparing Final Liquid Formulation of Different Dose Strength

| | Strength (amount of active | | |
|---|---|---|---|
| | drug, e.g. esomeprazole, | | |
| | per sachet) | | |
| | 2.5 mg | 10 mg | 40 mg |
| Amount of enteric coated. pellets*, | 10.6 | 42.6 | 170 |
| (in sachet) (mg) | | | |
| Amount of suspension modifying | 1 | 3 | 3 |
| granulate** (in same sachet as | | | |
| above) (g) | | | |
| Volume of water (ml) | 5 | 15 | 15 |

*made in accordance with Example 3.
**made in accordance with Example 1a.

### Example 5

### Illustration of the Rapid Gelling Time of the Present Invention

The content of a sachet containing the final formulation, having a 40 mg dose strength and prepared according to Ex. 4, was emptied into a beaker containing the nominally prescribed 15 ml amount of water.

The sample was then stirred for 15 seconds and then allowed to rest until 55 seconds from to start, after which it was again stirred for 5 seconds to evenly distribute the active drug granules in the suspension.

The suspension was inspected for 30 seconds to determine whether substantially all of the enteric coated pellets were distributed in the suspension or if they were assembled at the bottom of the beaker.

If the pellets were not distributed in the liquid medium but assembled at the bottom of the beaker, the process was repeated, i.e. waiting 25 seconds further and stirring 5 seconds, then waiting for 2 minutes, followed by inspecting for 30 seconds, until substantially all of the pellets remained distributed in the liquid medium. The time needed for the pellets to remain in suspension in the liquid medium was recorded.

The samples in the table below were evaluated in the described way, with the following results:

| | Time needed |
|---|---|
| | for pellets |
| Sample | to remain suspended |
| 1) Sachet cont. 40 mg dose strength, acc. to Ex. 4. | 2 minutes |
| 2) Sachet cont. 40 mg dose strength, acc. to Ex. 4. | 2 minutes |
| 2) Sachet cont. 10 mg dose strength, acc. to Ex. 4. | 2 minutes |
| I) Sachet “Lanzo ™ 30 mg” | 5 minutes |
| II) Sachet “Lanzo ™ 30 mg” | 5 minutes |

1. An oral pharmaceutical dosage form being a solid rapidly gelling granulate mixture, suitable for making a suspension comprising:
(I) an acid sensitive proton pump inhibitor being esomeprazole, an alkaline salt thereof, a hydrated form of esomeprazole, or a hydrated form of an alkaline salt of esomeprazole, as an active ingredient, distributed in a multitude of enteric coated pellets; and
(II) a granulate, wherein the granulate is a suspension modifying granulate comprising:
a rapidly dissolving diluent selected from the group consisting of glucose, sucrose, a hydrate of glucose and a hydrate of sucrose;
a gelling agent which is a xanthan gum;
an acidic pH-regulating agent;
a binder; and
optionally, a disintegrant, with the proviso that the granulate is free from bicarbonate and carbonate salts, and
wherein the ratio between the binder and the gelling agent in the granulate is from 1:2 to 1:3 w/w.

2. The dosage form according to claim 1, which is free from lactose.

3. The dosage form according to claim 1, wherein the suspension modifying granulate is obtained by mixing and granulating the rapidly dissolving diluent and the gelling agent together such that the rapidly dissolving diluent is randomly distributed throughout the obtained granulate particles.

4. The dosage form according claim 1, wherein the concentration of the gelling agent is 0.6% to 12% (w/w) of the suspension modifying granulate.

5. The dosage form according claim 1, wherein the concentration of the gelling agent is 1.8% to 4.8% (w/w) of the suspension modifying granulate.

6. The dosage form according to claim 1, wherein the suspension modifying granulate when suspended in water forms a suspension having a pH in the range of between 3.0 and 6.0.

7. The dosage form according to claim 1, wherein the suspension modifying granulate when suspended in water forms a suspension having a pH in the range of between 3.0 and 5.0.

8-13. (canceled)

14. The dosage form according to claim 1, wherein the enteric coated pellet consist of: a core material comprising the active ingredient, a subcoating layer, an enteric coating layer, with the proviso that the pellets do not have an additional coating layer on the enteric coating layer.

15. The dosage form according to claim 1, wherein the enteric coated pellets have an average diameter in the range of 0.2-1.8 mm.

16. The dosage form according to claim 1, wherein the enteric coated pellets have an average diameter in the range of 0.4-1.0 mm.

17. A sachet comprising the dosage form according to claim 1.

18. The sachet according to claim 17, wherein the amount of the proton pump inhibitor in the dosage form is in the range of 1 mg-100 mg.

19. The sachet according to claim 17, wherein the amount of the proton pump inhibitor in the dosage form is in the range of 1 mg-40 mg.

20. A ready-for-use liquid formulation comprising an aqueous liquid and the dosage form according to claim 1.

21. The liquid formulation according to claim 20, wherein the amount of the aqueous liquid is in the range of from 2.5 times up to 7.5 times the amount of the suspension modifying granulate.

22-25. (canceled)

26. The liquid formulation according to claim 20, wherein the aqueous liquid is water.

27. A process for preparing the suspension modifying granulate used in the dosage form according to claim 1, wherein the process comprises mixing together and granulating the rapidly dissolving diluent and the gelling agent together, and subsequently drying the obtained suspension modifying granulate, wherein the rapidly dissolving diluent is randomly distributed throughout the obtained individual granulate particles.

28. A process for preparing the suspension modifying granulate used in the dosage form according to claim 1, wherein the process comprises the steps in the following order:
I) mixing the gelling agent with the pH-regulating agent, the rapidly dissolving diluent, and the optional disintegrant;
II) dissolving the binder in ethanol;
III) wetting the mixture obtained in step I with the solution obtained in step II;
IV) agitating the wet mixture obtained in step III such that substantially each particle of the gelling agent is in contact with the rapidly dissolving diluent;
V) drying the agitated wet mixture from step IV until the final moisture content in the suspension modifying granulate measured as loss on drying is less than 3% (w/w); and
VI) grinding or milling the dry granules obtained in step V until more than 95% (w/w) of the granules pass through a sieve having 1.0 mm openings.

29. (canceled)

30. (canceled)

31. A method of treatment of gastric acid related diseases comprising administering an effective amount of an oral pharmaceutical dosage form as defined in claim 1 to a patient in need thereof.

32. (canceled)

33. A process for preparing the suspension modifying granulate used in the dosage form according to claim 1, wherein the process comprises the steps in the following order:
I) dissolving the binder in ethanol;
II) mixing the gelling agent with the pH-regulating agent, the rapidly dissolving diluent, and the optional disintegrant;
III) wetting the mixture obtained in step II with the solution obtained in step I;
IV) agitating the wet mixture obtained in step III such that substantially each particle of the gelling agent is in contact with the rapidly dissolving diluent;
V) drying the agitated wet mixture from step IV until the final moisture content in the suspension modifying granulate measured as loss on drying is less than 3% (w/w); and
VI) grinding or milling the dry granules obtained in step V until more than 95% (w/w) of the granules pass through a sieve having 1.0 mm openings.
